(19) United States
(12) Patent Application Publication
(10) Pub. No.: US 2009/0156412 A1
(43) Pub. Date: Jun. 18, 2009

(54) SURFACE-CAPTURE OF TARGET NUCLEIC ACIDS

(75) Inventors: John J. Boyce, IV, Bridgewater, MA (US); Timothy D. Harris, Toms River, NJ (US)

Correspondence Address: COOLEY GODWARD KRONISH LLP;ATTN: Patent Group, Suite 1100, 777 - 6th Street, NW, WASHINGTON, DC, 20001, US

(73) Assignee: HELICOS BIOSCIENCES CORPORATION, Cambridge, MA (US)

(21) Appl. No.: 11/958,173

(22) Filed: Dec. 17, 2007

(51) Int. Cl. C40B 20/02; C40B 50/06; C07H 21/00

(52) U.S. Cl. 506/3; 506/26; 536/24.3

(57) ABSTRACT

The disclosure provides methods of capturing target nucleic acids (e.g., gene or gene fragments) onto a solid support for further analysis. The disclosed methods utilize a capture probe that selectively circularizes only the target nucleic acid. Following the circularization of the target, the linear, non-target, nucleic acids are removed from the sample. Next, the circularized target is linearized and bound to a solid support. To allow for linearization, the capture probe may include a cleavage site that can be a noncanonical nucleotide(s) (e.g., uracil in DNA) and/or a rare-cutter site (e.g., the Not I restriction site). In some embodiments, the target nucleic acid is captured onto a support without an intermediate amplification step.

### TECHNICAL FIELD

The invention is in the field of molecular biology and relates to methods for nucleic acid analysis. In particular, the invention relates to methods of capturing target nucleic acids onto a solid support.

### BACKGROUND OF THE INVENTION

Many existing methods for nucleic acid analysis, including for example, gene sequencing, rely on selective amplification of the starting material by polymerase chain reaction (PCR), clonal amplification, or other amplification methods. These approaches are prone to the introduction of multiple replication errors that are inherent in the enzyme-based amplification methods. In contrast, recently developed sequencing technologies allow direct sequencing of a single nucleic acid molecule, thus eliminating any need for amplification of the starting material. As a result, such new methods yield a more reliable sequence output. For example, in true single-molecule sequencing (tSMS), an unamplified target nucleic acid is isolated from a sample and captured onto a solid support for further manipulation. For single-molecule sequencing, high specificity and capture efficiency are desirable. Low specificity may result in unacceptable background noise, while low efficiency may result in the loss of the target nucleic acid molecule.

Accordingly, a need exists for methods of selective and efficient capture of target nucleic acids onto a solid support for subsequent manipulation and analysis.

### SUMMARY OF THE INVENTION

The invention provides methods for robust selective capture of a target nucleic acid onto a solid support. Methods of the invention utilize a capture probe that selectively circularizes only the target nucleic acid. Following circularization of the target, the remaining linear (i.e., non-target) nucleic acids are removed from the sample. Next, the circularized target is linearized and bound to a solid support.

The invention provides methods for enriching a sample for the target molecules to be sequenced or otherwise manipulated. The methods therefore are useful for targeted sequencing or re-sequencing in a highly selective matter. The resulting support-bound population of nucleic acids is enriched for a selected target.

Methods of the invention are useful for manipulation of homogenous, as well as heterogeneous, populations of nucleic acids. Moreover, methods of the invention are especially amenable to multiplex reactions (e.g., single molecule sequencing methodologies) involving captured nucleic acids. As opposed to direct capture methods, the invention provides an efficient way of selecting for the target nucleic acid. In other aspects, the invention provides a method of sequencing a target nucleic acid, a method of determining a nucleic acid copy number, and other methods of analysis which require capturing a target nucleic acid onto a solid support using the methods of the invention.

Thus, according to the invention, methods comprise circularizing a target nucleic acid present in a sample, removing non-circularized nucleic acids, linearizing the target nucleic acid, and capturing the linearized target nucleic acid on a solid support. Preferred methods may additionally involve sample preparation techniques designed to obtain nucleic acids from cells. Such methods are known in the art and may include mechanical shearing, enzymatic digestion, etc.

For single-molecule sequencing, it is preferred that the circularized (target) nucleic acids be unamplified. However, for certain other contemplated embodiments, a user may amplify target nucleic acid by, for example, PCR, rolling circle amplification or any other standard amplification methods.

Capture of linearized target nucleic acids onto a solid support may be accomplished using hybrid capture techniques, non-specific binding (e.g., glass), or protein-based capture (e.g., by DNA- or RNA-binding proteins).

Capture probe comprises: 1) a double-stranded nucleic acid having two overhang ends that are specific (i.e., complementary) to two sites of the target nucleic acid, 2) one or more cleavage site(s) in the double-stranded region of the probe, and optionally 3) other elements. In certain embodiments, both overhang ends of the capture probe are complementary to restriction site(s) of a single or two different restriction enzymes used to isolate the target nucleic acid. The cleavage site that may be a noncanonical nucleotide(s) (such as, e.g., uracil in DNA) or a rare-cutter site (such as, e.g., the Not I restriction site). In some embodiments, the probe contains a capture sequence (e.g., polyNₙ, wherein N is U, A, T, G, or C, and n≧5).

Target nucleic acid may be linearized by any means, such as randomly fragmenting the linearized or circular single-stranded nucleic acid by shearing. In some embodiments, linearization is followed by adding a capture sequence to the linearized nucleic acid(s) (e.g., at the 3′ end(s)) and/or a recognition sequence (e.g., at the 5′ end(s)).

For example, target nucleic acids may be sequenced by conventional gel electrophoresis-based methods using, for example, Sanger-type sequencing. Alternatively, sequencing may be accomplished by use of several “next generation” methods that are not based upon the Sanger approach. In preferred embodiments, target nucleic acids are sequenced using a single-molecule sequencing-by-synthesis technique, as described in, e.g., a co-pending application published as U.S. Patent App. Pub. No. 2007/0070349. In such methods, the linearized target nucleic acid is hybridized to primers that are covalently attached to a derivatized glass surface so that a plurality of the resulting primer/target duplexes are individually optically resolvable. After a wash step, one or more optically labeled nucleotides is/are added along with a polymerase in order to allow template-dependent sequencing-by-synthesis to occur. The process is repeated until sufficient number of target nucleotides is determined. Sequencing may be conducted such that a single labeled species of nucleotides is added sequentially or multiple species with different labels are added at the same time. Other modifications of the process are contemplated as described in U.S. Pat. Nos. 7,282,337; 7,279,563; 7,276,720; 7,220,549; and 7,169,560.

### BRIEF DESCRIPTION OF THE FIGURES

FIG. 1 shows a schematic design of a capture probe used in the methods of the invention.

FIG. 2 is a diagram illustrating certain embodiments of the methods of the invention.

### DETAILED DESCRIPTION OF THE INVENTION

The invention provides methods of capturing nucleic acids onto a solid support. Upon capture, the nucleic acids are further manipulated or analyzed, e.g., by sequencing (e.g., exonic re-sequencing, genotyping, single nucleotide polymorphism (SNP) detection) or used for allele quantification, pathogen diagnostics, etc.

Methods of the invention utilize a capture probe that selectively circularizes the target nucleic acid. Following the circularization of the target, the linear (non-target) nucleic acids are removed from the sample. Next, the circularized target is linearized and bound onto a solid support.

Circular constructs for PCR-based amplification have been previously described (see, e.g., PCT Application Publication WO 2005/111236). Circularization of nucleic acids has been used to increase efficiency of PCR-based amplification of nucleic acids (see, e.g., Dahl et al. (2005) Nucleic Acid Res., 33, e71; and Dahl et al. (2007) Proc. Natl. Acad. Sci., 104:9387-9392). However, this approach has not been previously applied in the context of a single-molecule analysis, i.e., when the target is unamplified. In addition, the published methods describe circularization of relatively short fragments, typically, less than 200 nucleotides (nts). Thus, although the methods of the invention can be practiced with an additional step of amplification, in its preferred embodiments, the invention involves circularization of targets that are 300 nts or longer, preferably 500 nts or longer, followed by a capture of the unamplified nucleic acids.

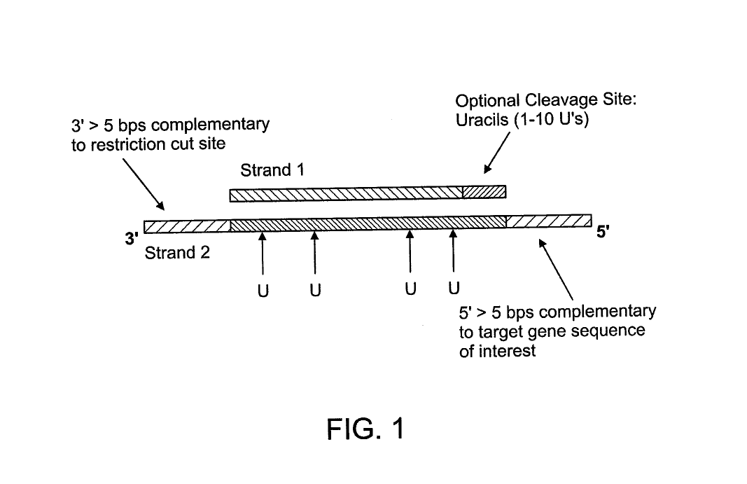

An example of a capture probe used in the methods of the invention is illustrated in FIG. 1. Generally, such a probe comprises: 1) a double-stranded nucleic acid having two overhang ends that are specific (i.e., complementary) to two sites of the target nucleic acid, 2) one or more cleavage site(s) in the double-stranded region of the probe, and 3) other optional elements. Various features of the capture probe are described in detail below.

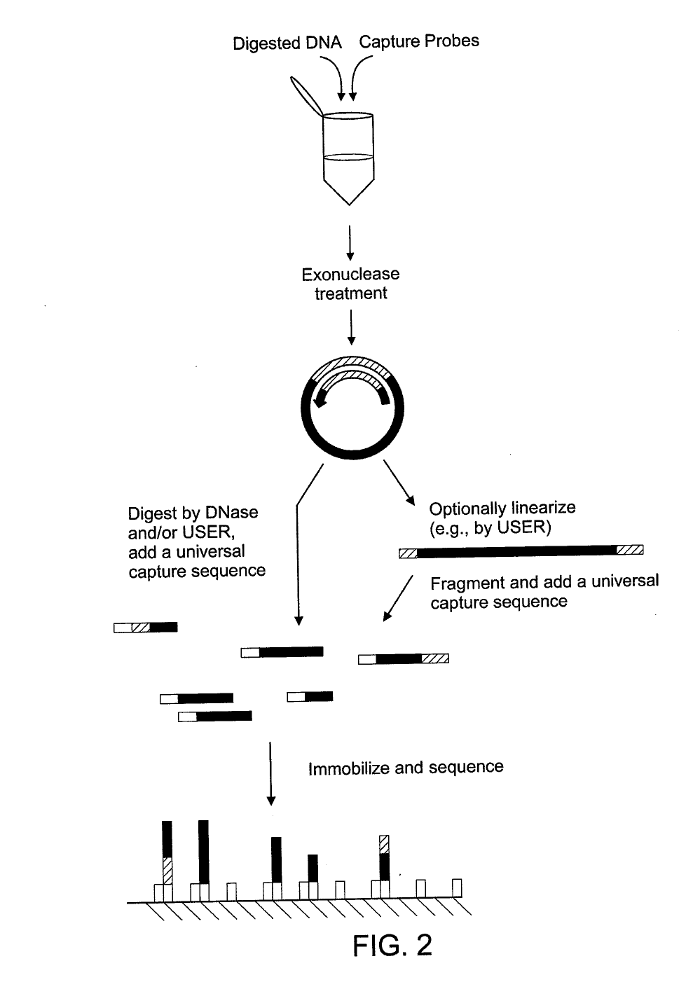

FIG. 2 illustrates certain embodiments of the methods for capturing target nucleic acid onto a solid support, according to the invention. Certain embodiments of the methods of the invention include the following steps:
(i) fragmenting a nucleic acid to produce one or more target fragments, each fragment having at least one defined end sequence;
(ii) denaturing the target fragment if it is double-stranded, thereby producing a single-stranded target fragment;
(iii) contacting the single-stranded target fragment with a double-stranded capture probe having two overhang ends specific to two corresponding sites of the target fragment;
(iv) allowing the capture probe and the target fragment to anneal to each other;
(v) optionally, cleaving any branched structures;
(vi) ligating the capture probe and the target fragment to form a closed circular nucleic acid;
(vii) removing remaining linear nucleic acids;
(viii) optionally, denaturing the double-stranded circular nucleic acid to create a single-stranded circular nucleic acid;
(ix) linearizing the single-stranded circular nucleic acid and, optionally, further fragmenting the linearized nucleic acid, or fragmenting the circular single-stranded nucleic acid;
(x) adding a capture sequence to the linearized nucleic acid fragment(s), and optionally adding a recognition site to the linearized nucleic acid fragment(s); and
(xi) capturing the linearized nucleic acids onto the solid support by hybridizing the capture sequence to a complementary sequence covalently attached to the solid support.

Target nucleic acid can come from a variety of sources. For example, nucleic acids can be naturally occurring DNA or RNA (e.g., mRNA or non-coding RNA) isolated from any source, recombinant molecules, cDNA, or synthetic analogs. For example, the target nucleic acid may include whole genes, gene fragments, exons, introns, regulatory elements (such as promoters, enhancers, initiation and termination regions, expression regulatory factors, expression controls, and other control regions), DNA comprising one or more single-nucleotide polymorphisms (SNPs), allelic variants, other mutations. The target nucleic acid may also be tRNA, rRNA, ribozymes, splice variants, antisense RNA, or siRNA.

Target nucleic acid may be obtained from whole organisms, organs, tissues, or cells from different stages of development, differentiation, or disease state, and from different species (human and non-human, including bacteria and virus). Various methods for extraction of nucleic acids from biological samples are known (see, e.g., Nucleic Acids Isolation Methods, Bowein (ed.), American Scientific Publishers, 2002). Typically, genomic DNA is obtained from nuclear extracts that are subjected to mechanical shearing to generate random long fragments. For example, genomic DNA may extracted from tissue or cells using a Qiagen DNeasy Blood & Tissue Kit following the manufacturer's protocols.

In order for the capture probe to anneal to the target nucleic acid, the probe should have at least one defined end that is complementary to one of the ends of the target; the other end of the probe should be complementary to the other end of the target or to a defined internal sequence flanking the target. As shown in FIG. 2, in the case of the probe having two ends complementary to sequences at the ends of the target nucleic acid, the probe and the target will anneal to form a noncovalently associated circular structure, whereas if one end of the probe is complementary to an internal sequence, the hybridization of the probe and the target will result in a branched structure. Multiple probes, each specific to a different target, can be used in a single multiplex reaction, thereby multiple targets can be captured and analyzed simultaneously.

To generate a target nucleic acid with at least one defined end sequence, the nucleic acid sample is treated with one more or more restriction enzymes. Restriction enzymes cleave nucleic acids at defined sites, thus producing fragments with defined end sequences. Any suitable restriction enzyme may be used to generate a target nucleic acid, so long as its recognition site falls outside of the region of interest. Consequently, as used herein, the term “target nucleic acid”, or “target”, refers to a region of interest and, as appropriate, includes flanking regions.

In preferred embodiments, the target nucleic acid has two defined ends that are unique to that target. Preferably, the probe contains two different defined ends corresponding to restriction sites of two different restriction enzymes that are used to isolate the target nucleic acid. A unique combination of defined ends (and thus the restriction enzymes to be used) can be identified for most targets, using for example, in silico methods (e.g., the PieceMaker program (Stenberg et al. (2005) Nucleic Acids Res., 33(8):e72); or using the NEBcutter tool available tools.neb.com/NEBcutter2/index.php). Alternatively, one may identify a unique combination of a defined end and a defined internal sequence.

The length of the target nucleic acid may vary. The average length of the target nucleic acid may be, for example, at least 300, 350, 400, 450, 500, 550, 600, 700, 800, 900, 1000, 2000, 3000, 4000, 5000 nts or longer. In some embodiments, the length of the target is between 300 and 5000 nts, 400 and 4000 nts, or 500 and 3000 nts.

In order to circularize the target nucleic acid, the following steps may be performed:
(ba) denaturing the target nucleic acid if it is double-stranded, thereby producing a single-stranded target nucleic acid;
(bb) contacting the single-stranded target nucleic acid with a capture probe having two overhang ends specific to two corresponding sites on the target fragment;
(bc) allowing the capture probe and the target nucleic acid to anneal to each other, thereby forming a noncovalently associated circular nucleic acid;
(bd) optionally, cleaving any branched structures, and
(be) ligating the capture probe and the target nucleic acid to form a partially double-stranded, covalently closed circular nucleic acid (cccNA).

Conditions for performing steps (ba) through (be) are generally known and may be adjusted depending on the nature of the target sequence and other parameters (see generally, e.g., Sambrook et al., Molecular Cloning: A Laboratory Manual, Cold Spring Harbor Laboratory Press, NY, Vol. 1, 2, 3 (1989)).

Step (ba) of denaturing the target nucleic acid to a single-stranded form involves subjecting the nucleic acid to denaturing conditions, such as high ionic strength, high temperature, high or low pH, etc. For example, the target nucleic acid can be denatured by being subjected to the temperature of 105° C. for 10-20 min.

Step (bc) of allowing the capture probe and the target nucleic acid to anneal to each other involves incubating the sample containing the target and the probe under conditions that are stringent enough to ensure specificity of hybridization, yet sufficiently permissive to allow formation of stable hybrids at an acceptable rate. The temperature and length of time required for probe/target annealing depend upon several factors including the base composition, length and concentration of the primer, and the nature of the solvent used, e.g., DMSO (dimethylsulfoxide), formamide, or glycerol, and counter-ions such as magnesium. Typically, hybridization (annealing) is carried out at a temperature that is approximately 5-10° C. below the melting temperature of the probe/target nucleic acid duplex in the annealing solvent. For example, the probe and the target can be annealed by gradually lowering the temperature of the sample from 95° C. to 45° C. over a period of 15-90 mins as illustrated in the Example.

The optional step (bd) of cleaving any branched structures may be performed prior to, or concurrently with, step (be). To cleave the branched structures, one may use Taq DNA polymerase (Thermus aquaticus) or one of the flap endonucleases (FENs), such as Mja nuclease (Methanococcus jannaschii), Tth polymerase (Thermus thermophilus), and Tfl polymerase (Thermus flavus), or another enzyme suitable for degrading branched structures.

Step (be) of ligating the capture probe and the target fragment is performed subsequent to the hybridization.

Following the circularization of the target nucleic acid, the linear nucleic acids remaining in the sample are removed (step (c)). The removal of linear nucleic acids can be accomplished by treating the sample with an exonuclease as described in, e.g., Dahl et al. (2005) Nucl. Acids Res., 33(8):1-7.

Following the removal of the linear nucleic acids, the target nucleic acid is linearized in step (d). The linearization can be accomplished in several ways, all which may be used individually or in combination, in any order. For example, the target may be linearized by mechanical shearing. For single molecule sequencing, the resulting random fragments should be of sufficient length to map back to a reference sequence. The sufficient length would depend on the complexity of the reference sequence, but in general, the fragments should be about 15-100 nts, for example, at least 15, 20, 25, 30, 35, 40 nts or longer.

In other embodiments, the target nucleic acid is linearized by treating the circularized target nucleic acid with one or more restriction enzymes that do not have a cut-site in the target nucleic acid. In some embodiments, the circularized target nucleic acid is cut with a rare-cutter restriction enzyme (“rare-cutter”). In such embodiments, the rare-cutter's recognition site is incorporated into the probe by design. A rare-cutter is an enzyme whose restriction site is unlikely to be present within the target nucleic acid. Generally, a rare-cutter restriction enzyme is a restriction enzyme whose recognition site is rare in a given genome. For example, for the human genome, restriction enzymes whose recognition sites occur on average every 50,000 base pars (bps) or less frequently (e.g., every 100,000 bps or less frequently, 200,000 bps or less frequently, 500,000 bps or less frequently) would be considered rare-cutters. Examples of rare-cutter restriction enzymes and their respective recognition sites that can be used in the present invention include Not I and other enzymes shown in Table 1. Other rare-cutter enzymes can be found in, e.g., Restriction Endonucleases (Nucleic Acids and Molecular Biology) by Pingoud (Editor), Springer; 1 ed. (2004)). Many rare-cutter enzymes are available commercially, e.g, from New England BioLabs (Beverly, Mass.).

TABLE 1

| Restriction | Recognition | Frequency in Human |
|---|---|---|
| Enzyme | Site | Genome (bps) |
| Not I | GCGGCCGC | 1,000,000 |
| Xma III | CGGCCG | 100,000 |
| Sst II | CCGCGG | 100,000 |
| Sal I | GTCGAC | 100,000 |
| Nru I | TCGCGA | 300,000 |
| Nhe I | GCTAGC | 100,000 |

In those embodiments that utilize a capture probe with a cleavage site containing noncanonical nucleotides, the target nucleic acid may be linearized with a glycosylase-lyase and an endonuclease. In some embodiments, a basic site(s) is/are present in the probe before the circularization. In such a case, only an endonuclease is necessary to cleave the probe.

In such a case, a glycosylase-lyase specific to the noncanonical base excises the noncanonical base(s), leaving an a basic site(s), thereupon the endonuclease cleaves the phosphodiester bond at the a basic site(s). For example, if the target nucleic acid is DNA, one or more (e.g., 1-15) uracil residues may be incorporated into the probe. The construct is then linearized by the treatment with uracil N-glycosylase (UNG) and endonuclease IV. These enzymes and related reagents are available commercially, e.g., Uracil-DNA Excision Mix from Epicenter (Cat. No. UEM04100, Madison, Wis.) and the USER reagent from New England BioLabs (Cat. No. E5500S, Ipswich, Mass.). Other noncanonical nucleotides and respective glycosylases may be used as described below (see esp. Table 2 below; see also Demple et al. (1994) Annual Rev. Biochem., 63:915-948, and Lindahl (1979) Progress in Nucl. Acids Res., 22:135-192). Circular nucleic acid may be double-stranded or may be denatured to a single-stranded nucleic acid these enzymes.

As a result of the fragmentation, a capture sequence and/or a recognition site may be absent in all or some fragments. If so, these elements can be added after the fragmentation. Accordingly, in some embodiments, step (d) of linearizing the target nucleic acid is followed by adding a capture sequence to the linearized nucleic acid(s) at the 3′ end(s) of the target or target's fragments. Similarly, step (d) may also be followed by adding a recognition site to the linearized nucleic acid(s), e.g., at the 5′ end(s) of the target or target's fragments.

The capture sequence, also referred to as a universal capture sequence, is a nucleic acid sequence complimentary to a sequence attached to a solid support and may also include a universal primer. Depending on the target nucleic acid, the primer may comprise DNA, RNA or a mixture of both. In some embodiments, the linearized nucleic acids are bound onto the solid support by hybridizing the capture sequence to a complementary sequence covalently attached to the solid support. In some embodiments, the capture sequence is polyNₙ, wherein N is U, A, T, G, or C, n≧5, e.g., 10-30, 15-25, e.g., about 20. For example, the capture sequence could be polyA₂₀₋₃₀ or its complement.

As an alternative to a capture sequence, a member of a coupling pair (such as, e.g., antibody/antigen, receptor/ligand, or the avidin-biotin pair as described in, e.g., U.S. Patent Application No. 2006/0252077) may be linked to each fragment to be captured on a surface coated with a respective second member of that coupling pair.

The recognition site at the 5′ end of the sequence may be a second primer sequence that is used for re-sequencing following the “melt-and-resequence” procedure as described in U.S. Pat. No. 7,283,337.

In some embodiments, the circularized target nucleic acid is linearized solely by a cut within the probe, thus creating a linearized target nucleic acid of a uniform length (i.e., without further random fragmenting). In such a case, a universal capture sequence and/or a recognition site can be incorporated directly into the probe, which then makes it unnecessary to add these elements following the linearization.

In the next step, the linearized target nucleic acid is bound to a solid support. In preferred embodiments, the support-bound target nucleic acid is unamplified relative to its state prior to the circularization. In other embodiments, the target sequence may be amplified prior to capture onto the solid support, e.g., by using one of the following amplification methods: the polymerase chain reaction (PCR), and the ligase chain reaction (LCR), both of which require thermal cycling, the transcription based amplification system (TAS), the nucleic acid sequence based amplification (NASBA), the strand displacement amplification (SDA), the invader assay, rolling circle amplification (RCA), and hyper-branched RCA (HRCA).

The solid support may be, for example, a glass surface such as described in, e.g., U.S. Patent App. Pub. No. 2007/0070349. The surface may be coated with an epoxide, polyelectrolyte multilayer, or other coating suitable to bind nucleic acids. In preferred embodiments, the surface is coated with epoxide and a complement of the capture sequence is attached via an amine linkage. The surface may be derivatized with avidin or streptavidin, which can be used to attach to a biotin-bearing target nucleic acid. Alternatively, other coupling pairs, such as antigen/antibody or receptor/ligand pairs, may be used. The surface may be passivated in order to reduce background. Passivation of the epoxide surface can be accomplished by exposing the surface to a molecule that attaches to the open epoxide ring, e.g., amines, phosphates, and detergents.

Subsequent to the capture, the sequence may be analyzed, for example, by single molecule detection/sequencing, e.g., as described in the Example and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide may be incorporated and multiple lasers may be utilized for stimulation of incorporated nucleotides.

Accordingly, in one aspect, the invention provides a method of sequencing a nucleic acid, comprising sequencing the linearized nucleic acid that is captured onto a solid support in accordance with the methods described above. In another aspect, the invention provides a method of determining a nucleic acid copy number, comprising capturing an unamplified target nucleic acid onto a solid surface using methods of the invention and determining the number of the captured target nucleic acids, for example, by reference to a known control. The known control used as a reference might be either endogenous or exogenous. For example, one may select one or more genes within the sample (known single copy abundance) and relate the relative ratio or abundance of other genes to the control(s). Alternatively, for an exogenous control, one may add in a known amount of a nucleic acid sequence which is not naturally occurring in the sample and relate the relative ratio or abundance of other genes to this external control.

### Features of The Capture Probe

A capture probe, according to the invention, comprises: 1) a double-stranded nucleic acid having two overhang ends that are specific to two sites of the target nucleic acid, 2) one or more cleavage site(s) in the double-stranded region of the probe, and optionally 3) other elements. The size of the double-stranded region of the probe may vary. The minimum double-stranded structure should be sufficient to include a cleavage site and to allow efficient ligation with a target nucleic acid, as well as to incorporate any optional elements in the design of the probe. Typically, the double-stranded part of the probe is about 3-50 bps long, e.g., 3-30, 5-25, 10-40, 20-50, 25-40, or 30-40 bps.

The overhang ends are typically about 3-60 nucleotides each, e.g., 5-25, 10-40, 25-60, 20-50, 30-40, 19, 18, 17, 16, 15, 14, 10 nts, however, longer or shorter overhang ends can be used. The specific end sequences and the length of the overhang ends are chosen based on the restriction enzymes (and/or that target's internal sequence) used to isolate the target nucleic acid.

The cleavage site is located within the double-stranded portion of the capture probe and may include a noncanonical nucleotide(s) and/or a rare-cutter restriction enzyme recognition site(s). The noncanonical nucleotides should be incorporated in that strand on the probe which is to be ligated to the target nucleic acid. Examples of noncanonical nucleotide(s) include uracil for DNA and other nucleotides as shown in Table 2 in U.S. Pat. No. 6,190,865, which is reproduced below.

TABLE 2

| | | Non- | | | |
|---|---|---|---|---|---|
| Canonical | Non- | Canonical | | | |
| Base in | Canonical | Nucleotide | | Source of | Glycosylase |
| DNA | Nucleotide | Reference | Glycosylase | Glycosylase | Reference |
| T (thymine) | dUTP | Bessmans et | UDG or UNG | E. coli | Lindahl, |
| | (deoxyuridine | al., 1958 | | | 1974 |
| | triphosphate) | | | | |
| G | dITP | Thomas et al., | HXNG | a) calf | Karran and |
| (guanine) | (deoxyinosine | 1978 | (hypoxanthine- | thymus; | Lindahl, |
| | triphosphate) | | N-glycosylase) | b) E. coli | 1980, 1978 |
| C | 5-OHMe- | Stahl and | hydroxy- | calf | Cannon et |
| (cytosine) | dCTP | Chamberlin, | methyl | thymus | al., 1988 |
| | (5-hydroxy- | 1976 | cytosine-N- | | |
| | methyl | | glycosylase | | |
| | deoxycytidine | | | | |
| | triphosphate) | | | | |

The uracil-containing cleavage site may, for example, contain 1-10 uracils (e.g., 2-8, 3-6, 2, 3, 4, 5, 6, 7, 8, 9, and 10 or more uracil residues). In the case of a large number of uracils, an adaptor sequence may be used at one or both ends of the uracil region to increase the stability of the probe.

As illustrated in FIG. 1, uracils may be present in both strands of the double-stranded probe to simultaneously achieve the linearization as well as degradation of the second strand of the probe. In more specific embodiments, the shorter strand of the probe (Strand 1 as per FIG. 1) contains one uracil cleavage site containing, for example, 1-10 uracils. This site may be located equidistantly from both ends of the probe or proximally to one of the ends, preferably, towards the 3′ end of Strand 1, e.g., within the 3′ quartile of Strand 1. Additional cleavage sites, including uracil cleavage sites may be incorporated into Strand 1. Strand 2 may also contain one or more uracil cleavage sites (each site containing 1-10 uracils) dispersed throughout the strand. For example, Strand 2 may contain 2, 3, 4, 5, 6, 7 or more uracil cleavages sites. In certain embodiments, the linearized nucleic acid may be fragmented (e.g., by mechanical shearing) into smaller fragments of sufficient length. In some embodiments, the probe comprises at least 1 uracil cleavage site in each strand of the double-stranded probe.

Examples of rare-cutter recognition sites and respective restriction enzymes are shown in Table 1. Accordingly, in some embodiments, the capture probe comprises one or more sequences from Table 1. Other rare-cutter sites may be used as discussed above. Selection of the appropriate site will depend, in part, on the target nucleic acid, e.g., whether or not a particular restriction site is expected to be present in the target.

In some embodiments, the capture probe is a DNA that comprises one or more uracils and one or more rare-cutter sites (e.g., the Not I site).

The position of the cleavage site within the probe may vary. In some embodiments, the cleavage site is located approximately equidistantly from either end of the probe. In some embodiments, the site is located at the 3′ end of the capture sequence, while the capture sequence would be located at 3′ end of the target nucleic acid upon ligation, as illustrated in FIG. 1.

In some embodiments, additional optional features may be incorporated into the capture probe. Such features include, for example, one or two universal primer sequences that may be incorporated at one or both ends of the cleavage site, a probe-specific “bar-code” sequence, or other elements. In amplification-free embodiments, the probe need not include PCR primers.

Accordingly, in certain embodiments, the invention provides a nucleic acid probe comprising:
(a) a double-stranded nucleic acid having two overhang ends specific to two sites on a target nucleic acid, with one overhang end being complementary to a restriction cut site flanking a target sequence and the other end being complementary to a restriction cut site or an internal sequence;
(b) a cleavage site within the double-stranded nucleic acid of (a), said cleavage site selected from noncanonical nucleotide(s) and a rare-cutter site; and
(c) a capture sequence.

Probes can be synthetically made using conventional nucleic acid synthesis techniques. For example, probes may be synthesized on an automated DNA synthesizer (e.g., Applied Biosystems, Foster City, Calif.) using standard chemistries, such as phosphoramidite chemistry.

The following Example provides illustrative embodiments of the invention and does not in any way limit the invention.

### EXAMPLE

Genomic DNA is extracted from cultured cells by using the DNeasy Blood & Tissue Kit (Qiagen) or the Gentra genomic DNA preparation kit (Minneapolis, Minn.) following the manufacturers' protocols.

10 units of a restriction enzyme are used to digest the genomic DNA in manufacturer's recommended buffer and temperature for 1 hour to a final concentration of 100 ng/μl. To denature the digested DNA before the circularization reaction, the samples are heated to 95° C. for 15 min by using a thermal cycler. 250 ng of DNA is added to a capture probe in a total concentration of 10 nM, 100 nM of the uracil-containing probe, 1× Ampligase buffer (Epicentre, Madison, Wis.), 1 mM NAD, 5 units of Taq DNA polymerase (Invitrogen, Carlsbad, Calif.), 2 mM MgCl₂, and 5 units of Ampligase (Epicentre) to a final volume of 20 μl. The mixture is incubated at 95° C. for 10 min, followed by 75° C. for 15 min, 65° C. for 15 min, 55° C. for 15 min, and 45° C. for 15 min.

The circularized target is linearized by the addition of Uracil DNA-Excision Reagent (Epicenter) as per manufacturer's instructions. Specifically, 10 μl of the circularization reaction mix is combined with 10-μl mixtures of 1× Uracil Excision Buffer (Epicentre), 5 mM MgCl₂, 0.01 μg/μl BSA, and 1 μl Uracil-Excision Mix (Epicentre) and incubated for 1 hour at 37° C. followed by 80° C. for 20 min.

The linearized probe-target construct is then randomly fragmented by treatment with DNase I (New England BioLabs) to yield fragments of sufficient length to map back to a reference sequence, typically, 40-200, e.g., about 180 nts. Specifically, approximately 25 μg of DNA is digested with 0.1 U DNase I by incubating for 10 minutes at 3° C. Digested DNA fragment sizes are estimated by running an aliquot of the digestion mixture on a precast denaturing (TBE-Urea) 10% polyacrylamide gel (Novagen) and staining with SYBR Gold (Invitrogen/Molecular Probes). The DNase 1-digested DNA is filtered through a YM10 ultrafiltration spin column (Millipore) to remove small digestion products less than about 30 nt.

Approximately 20 pmol of the filtered DNase I digest is then polyadenylated with terminal transferase according to known methods (Roychoudhury et al., Terminal transferase-catalyzed addition of nucleotides to the 3′ termini of DNA. (1980) Methods Enzymol., 65(1):43-62).

An average of 50 A bases are added to each target, followed by addition of a ddNTP to terminate the target. The ddNTP may include a detectable label (fluorophore, e.g. Cy3) to monitor the attachment to the surface. These polyA-tailed target fragments are then captured onto a sequencing surface specially prepared for this test.

Epoxide-coated glass slides are prepared for oligo attachment. Epoxide-functionalized 40 mm diameter #1.5 glass cover slips (slides) are obtained from Erie Scientific (Salem, N.H.). The slides are preconditioned by soaking in 3×SSC for 15 minutes at 37° C. Next, a 500-pM aliquot of 5′ aminated oligo-dT50 is incubated with each slide for 30 minutes at room temperature in a volume of 80 ml. The slides are then treated with phosphate (1 M) for 4 hours at room temperature in order to passivate the surface. Slides are then stored in 20 mM Tris, 100 mM NaCl, 0.001% Triton X-100, pH 8.0 at 4° C. until they are used for sequencing.

For sequencing, the slide is placed in a modified FCS2 flow cell (Bioptechs, Butler, Pa.) using a 50-μm thick gasket. The flow cell is placed on a movable stage that is part of a high-efficiency fluorescence imaging system built based on a Nikon TE-2000 inverted microscope equipped with a total internal reflection (TIR) objective. The slide is then rinsed with HEPES buffer with 100 mM NaCl and equilibrated to a temperature of 50° C. An aliquot of the nucleic acid fragments prepared as described above is diluted in 3×SSC to a final concentration of 1.2 nM. A 100-μl aliquot is placed in the flow cell and incubated on the slide for 15 minutes. After incubation, the flow cell is rinsed with 1×SSC/HEPES/0.1% SDS followed by HEPES/NaCl. A passive vacuum apparatus is used to pull fluid across the flow cell. The resulting slide contains target/oligo(dT) primer template duplex. The temperature of the flow cell is then reduced to 37° C. for sequencing and the objective is brought into contact with the flow cell.

Further, cytosine triphosphate, guanidine triphosphate, adenine triphosphate, and uracil triphosphate, each having a cleavable cyanine-5 label (at the 7-deaza position for ATP and GTP and at the C5 position for CTP and UTP (PerkinElmer)) are stored separately in buffer containing 20 mM Tris-HCl, pH 8.8, 50 μM MnSO₄, 10 mM (NH4)₂SO₄, 10 mM HCl, and 0.1% Triton X-100, and 100 U Klenow exo⁻ polymerase (NEB). Sequencing proceeds as follows.

First, initial imaging is used to determine the positions of duplex on the epoxide surface. The Cy3 label attached to the nucleic acid fragments is imaged by excitation using a laser tuned to 532 nm radiation (Verdi V-2 Laser, Coherent, Santa Clara, Calif.) in order to establish duplex position. For each slide only single fluorescent molecules that are imaged in this step are counted. Imaging of incorporated nucleotides as described below is accomplished by excitation of a cyanine-5 dye using a 635-nm radiation laser (Coherent). 100 nM Cy5-CTP is placed into the flow cell and exposed to the slide for 2 minutes. After incubation, the slide is rinsed in 1×SSC/15 mM HEPES/0.1% SDS/pH 7.0 (“SSC/HEPES/SDS”) (15 times in 60 μl volumes each, followed by 150 mM HEPES/150 mM NaCl/pH 7.0 (“HEPES/NaCl”) (10 times at 60 μl volumes). An oxygen scavenger containing 30% acetonitrile and scavenger buffer (134 μl 150 mM HEPES/100 mM NaCl, 24 μl 100 mM Trolox in 150 mM MES, pH 6.1, 10 μl 100 mM DABCO in 150 mM MES, pH 6.1, 8 μl 2M glucose, 20 μl 50 mM NaI, and 4 μl glucose oxidase (USB) is next added. The slide is then imaged (500 frames) for 0.2 seconds using an Inova 301K laser (Coherent) at 647 nm, followed by green imaging with a Verdi V-2 laser (Coherent) at 532 nm for 2 seconds to confirm duplex position. The positions having detectable fluorescence are recorded. After imaging, the flow cell is rinsed 5 times each with SSC/HEPES/SDS (60 μl) and HEPES/NaCl (60 μl). Next, the cyanine-5 label is cleaved off incorporated CTP by introduction into the flow cell of 50 mM TCEP for 5 minutes, after which the flow cell is rinsed 5 times each with SSC/HEPES/SDS (60 μl) and HEPES/NaCl (60 μl). The remaining nucleotide is capped with 50 mM iodoacetamide for 5 minutes followed by rinsing 5 times each with SSC/HEPES/SDS (60 μl) and HEPES/NaCl (60 μl). The scavenger is applied again in the manner described above, and the slide is again imaged to determine the effectiveness of the cleave/cap steps and to identify non-incorporated fluorescent objects.

The procedure described above is then conducted 100 nM Cy5-dATP, followed by 100 nM Cy5-dGTP, and finally 100 nM Cy5-dUTP. Uridine may be used instead of Thymidine due to the fact that the Cy5 label is incorporated at the position normally occupied by the methyl group in Thymidine triphosphate, thus turning the dTTP into dUTP. The procedure (expose to nucleotide, polymerase, rinse, scavenger, image, rinse, cleave, rinse, cap, rinse, scavenger, final image) is repeated from 40 to 120 cycles.

Once a desired number of cycles are completed, the image stack data (i.e., the single-molecule sequences obtained from the various surface-bound duplex) are aligned to the reference sequence. The sequence data obtained is compressed to collapse homopolymeric regions. For example, the sequence “TCAAAGC” would be represented as “TCAGC” in the data tags used for alignment. Similarly, homopolymeric regions in the reference sequence are collapsed for alignment. The sequencing protocol described above results in an aligned sequence with an accuracy of between 98.8% and 99.96% (depending on depth of coverage). The individual single molecule sequence read lengths obtained range from 2 to 33 consecutive nucleotides with about 12.6 consecutive nucleotides being the average length. Other details of the protocol are described in process as described, for example, in U.S. Patent Application Publications Nos. 2007/0070349 and 2006/0252077.

All publications, patents, patent applications, and biological sequences cited in this disclosure are incorporated by reference in their entirety.

1. A method of capturing a target nucleic acid onto a solid support, the method comprising:
(a) obtaining a sample comprising a target nucleic acid;
(b) circularizing the target nucleic acid;
(c) removing non-circularized nucleic acids;
(d) linearizing the target nucleic acid; and
(e) capturing the linearized target nucleic acid onto the solid support.

2. The method of claim 1, wherein the linearized target nucleic acid which is captured onto the solid support is unamplified.

3. The method of claim 1, wherein step (a) of obtaining the target nucleic acid comprises fragmenting a starting nucleic acid to produce the target nucleic acid having at least one defined end sequence.

4. The method of claim 3, wherein the average length of the target nucleic acid is at least 500 nts.

5. The method of claim 3, wherein the target nucleic acid contains (1) a unique combination of two defined ends or (2) a unique combination of one defined end sequence and one internal sequence.

6. The method of claim 1, wherein step (aa) comprises digesting the starting nucleic acid with one or more restriction enzymes.

7. The method of claim 1, wherein step (b) of circularizing the target nucleic acid comprises:
(ba) denaturing the target nucleic acid if it is double-stranded, thereby producing a single-stranded target nucleic acid.
(bb) contacting the single-stranded target nucleic acid with a double-stranded capture probe having two overhang ends specific to two corresponding sites on the target nucleic acid;
(bc) allowing the capture probe and the target nucleic acid to anneal to each other;
(bd) optionally, cleaving any branched structures; and
(be) ligating the capture probe and the target fragment to form a partially double-stranded closed circular nucleic acid.

8. The method of claim 7, wherein both overhang ends of the capture probe are complementary to two respective restriction cut sites of two different restriction enzymes.

9. The method of claim 1, wherein step (c) of removing the linear nucleic acids comprises treating the linear nucleic acids with an exonuclease.

10. The method of claim 1, wherein step (d) of linearizing the target nucleic acid comprises treating the circularized target nucleic acid with a rare-cutter restriction enzyme.

11. The method of claim 1, wherein step (d) of linearizing the target nucleic acid comprises treating the circularized target nucleic acid with glycosylase-lyase and endonuclease.

12. The method of claim 1, wherein step (d) of linearizing the target nucleic acid comprises treating the circularized target nucleic acid with uracil DNA glycosylase-lyase and endonuclease VIII.

13. The method of claim 1, wherein step (d) of linearizing the target nucleic acid comprises randomly fragmenting the linearized or circular single-stranded nucleic acid by shearing.

14. The method of claim 13, wherein the random fragments produced are of sufficient length to map back to a reference sequence.

15. The method of claim 1, wherein step (d) of linearizing the target nucleic acid is followed by adding a capture sequence to the linearized nucleic acid(s) at the 3′ end(s) if the capture sequence is absent.

16. The method of claim 15, wherein the capture sequence is polyNₙ, wherein N is U, A, T, G, or C, and n≧5.

17. The method of claim 1, wherein step (d) of linearizing the target nucleic acid is followed by adding a recognition site to the linearized nucleic acid(s) at the 5′ end(s) if the recognition site is absent.

18. The method of claim 1, wherein in step (e) the linearized nucleic acids are bound onto the solid support by hybridizing the capture sequence to a complementary sequence covalently attached to the solid support.

19. A method of sequencing a nucleic acid, comprising:
(i) capturing a target nucleic acid onto a solid support using the method of claim 1; and
(ii) sequencing the linearized nucleic acids captured on the solid support.

20. A method of determining a nucleic acid copy number, comprising:
(i) capturing an unamplified target nucleic acid onto a solid support using the method of claim 1; and
(ii) determining the copy number of the linearized nucleic acids captured on the solid support.

21. A method of capturing a nucleic acid onto a solid support, the method comprising:
(i) fragmenting a nucleic acid to produce one or more target fragments, each fragment having at least one defined end sequence;
(ii) denaturing the target fragment if it is double-stranded, thereby producing a single-stranded target fragment;
(iii) contacting the single-stranded target fragment with a double-stranded capture probe having two overhang ends specific to two corresponding sites on the target fragment;
(iv) allowing the capture probe and the target fragment to anneal to each other;
(v) optionally, cleaving any branched structures;
(vi) ligating the capture probe and the target fragment to form a closed circular nucleic acid;
(vii) removing remaining linear nucleic acids;
(viii) optionally, denaturing the double-stranded circular nucleic acid to create a single-stranded circular nucleic acid;
(ix) linearizing the single-stranded circular nucleic acid and, optionally, further fragmenting the linearized nucleic acid, or fragmenting the circular single-stranded nucleic acid;
(x) adding a capture sequence at the 3′ end(s) of the linearized nucleic acid fragment(s), and optionally adding a recognition site at the 5′ end(s) of the linearized nucleic acid fragment(s); and
(xi) capturing the linearized nucleic acids onto the solid support by hybridizing the capture sequence to a complementary sequence covalently attached to the solid support.

22. A nucleic acid probe comprising:
(a) a double-stranded nucleic acid having two overhang ends specific to two sites on a target nucleic acid, with one overhang end being complementary to a restriction cut site flanking a target sequence and the other end being complementary to a restriction cut site or an internal sequence;
(b) a cleavage site within the double-stranded nucleic acid of (a), said cleavage site selected from noncanonical nucleotide(s) and a rare-cutter site; and
(c) a capture sequence.

23. The probe of claim 22, wherein the capture sequence is polyNₙ, wherein N is U, A, T, G, or C, and n≧5.

24. The probe of claim 22, wherein the cleavage site comprises 1-10 uracils.

25. The probe of claim 22, wherein the probe comprises at least 1 uracil cleavage site in each strand of the double-stranded probe.
